(19) United States
(12) Patent Application Publication
(10) Pub. No.: US 2021/0177300 A1
(43) Pub. Date: Jun. 17, 2021

(54) MONITORING ABNORMAL RESPIRATORY EVENTS

(71) Applicant: KONINKLIJKE PHILIPS N.V., EINDHOVEN (NL)

(72) Inventor: Ali Akbar Ahmad SAMADANI, Somerville, MA (US)

(21) Appl. No.: 17/120,350

(22) Filed: Dec. 14, 2020

Related U.S. Application Data
(60) Provisional application No. 62/949,067, filed on Dec. 17, 2019.

(51) Int. Cl. A61B 5/08

(52) U.S. Cl. CPC A61B 5/0823; A61B 5/0816

(57) ABSTRACT

Proposed are concepts for monitoring abnormal respiratory events of a subject which leverage data from a sensor system. Proposed concepts may employ data acquisition from the sensor system at two different frequencies. For instance, a first, lower frequency may be used to acquire data from which an abnormal respiratory event (such as a cough or wheeze) of the subject may be detected. In response to detecting an abnormal respiratory event, a second, higher frequency may be used to acquire data to facilitate more detailed analysis and/or monitoring of the subject's respiration. In this way, low frequency data acquisition, which may be less accurate but consume less power, may be used to firstly detect an abnormal respiratory event. Once an abnormal respiratory event detected, data acquisition may be switched to a higher frequency, so as to obtain more detailed (e.g. higher resolution) information about the respiration.

### CROSS-REFERENCE TO PRIOR APPLICATIONS

This application claims the benefit of U.S. Provisional Application No. 62/949,067, filed on 17 Dec. 2019. This application is hereby incorporated by reference herein.

### FIELD OF THE INVENTION

The invention relates to monitoring a subject, and more particularly to monitoring abnormal respiratory events of a subject using a sensor system.

### BACKGROUND OF THE INVENTION

Abnormal respiratory events (e.g. a cough, wheezing, shortened breath, dyspnea, trepopnea, etc.) may be symptomatic of respiratory disease or illness, such as a Chronic Lower Respiratory Disease (CLRD) (e.g. Chronic Obstructive Pulmonary Disease (COPD), asthma, and pulmonary hypertension).

Although abnormal respiratory events can occur at any time and at any location, current practices for monitoring and assessing respiratory health of a subject are limited to clinical visits. This can result in the diagnosis and/or treatment of respiratory disease or illness of a subject being missed or delayed. Because early detection of symptoms (or the worsening thereof) can reduce usage of emergency medical resources and/or improve outcomes, there is a need to be able to monitor abnormal respiratory events of a subject. Subject monitoring systems currently exist, but they can be intrusive, difficult to use, inccurate and/or have limited functionality.

### SUMMARY OF THE INVENTION

The invention is defined by the claims.

According to examples in accordance with an aspect of the invention, there is provided a computer-implemented method for monitoring abnormal respiratory events of a subject using a sensor system configured to detect respiration of the subject and generate respiration data representative of the detected respiration. The method comprises: controlling the sensor system to detect respiration of the subject at a first detection frequency; obtaining first respiration data from the sensor system, the first respiration data being representative of respiration of the subject detected at the first detection frequency; detecting an abnormal respiratory event of the subject based of the first respiration data; responsive to detecting an abnormal respiratory event of the subject, controlling the sensor system to detect respiration of the subject at a second detection frequency, the second detection frequency being higher than the first detection frequency; and obtaining second respiration data from the sensor system, the second respiration data being representative of respiration of the subject detected at the second detection frequency.

Proposed are concepts for monitoring abnormal respiratory events of a subject which leverage data from a sensor system. Such a sensor system may be used to obtain data from which respiration of the subject may be detected and analyzed. Proposed concepts may employ data acquisition from the sensor system at two different frequencies. For instance, a first, lower frequency may be used to acquire data from which an abnormal respiratory event (such as a cough or wheeze) of the subject may be detected. In response to detecting an abnormal respiratory event, a second, higher frequency may be used to acquire data to facilitate more detailed analysis and/or monitoring of the subject's respiration. In this way, low frequency data acquisition, which may be less accurate but consume less power, may be used to firstly detect an abnormal respiratory event. Once an abnormal respiratory event is detected, data acquisition may be switched to a higher frequency, so as to obtain more detailed (e.g. higher resolution) information about the respiration. Such a proposal may save power whilst retaining data accuracy, e.g. by only employing a high frequency (i.e. high accuracy) data acquisition mode in response to detecting an abnormal respiratory event.

Embodiments may thus use data from conventional or existing subject monitoring systems. Such systems need not employ specialized respiration sensors, since an abnormal respiratory event may be detected based on various different types of monitored parameters or data. For example, accelerometer data from an activity tracking device or a sternum-worn vibration sensor may be analyzed to detect an abnormal respiratory event. For instance, detected movement or vibration of a body part of a subject may exhibit a specific trait or pattern when the subject coughs or wheezes. By way of another example, the sound of a cough or wheeze may be detected from sound data generated by an audio capture device (e.g. microphone) that carried or worn by the subject. Proposed embodiments may thus provide an additional level of monitoring without requiring additional, specialized monitoring devices to be employed. Instead, existing devices may be used by proposed embodiments.

Proposed embodiments may therefore facilitate monitoring of abnormal respiratory events using conventional devices, and such monitoring may have reduced (or minimal) power requirements whilst providing accurate analysis and/or monitoring of detected abnormal respiratory events. Improved and more robust detection, analysis and monitoring of abnormal respiratory events may thus be provided by embodiments.

Embodiments may thus enable routine objective, in-home, and low-burden monitoring of abnormal respiratory events of a subject, and this may facilitate prompt and effective medical treatment/intervention (which may be particularly important for high-risk subjects).

Purely by way of example, an embodiment may control a chest-worn/chest-affixed inertial measurement unit (IMU) to track chest vibrations of a subject at a first, low frequency. Based on such tracked vibrations, an abnormal respiratory event may be detected and, responsive to such detection, the IMU may then be controlled to track chest vibrations of a subject at a second, higher frequency. Vibrations tracked at the second frequency may provide detailed and accurate information about the respiration of the subject that may facilitate an improved analysis and understanding of the subject's respiratory condition. This may provide for improved delivery of care and well-informed clinical decision making.

It will therefore be appreciated that improved Clinical Decision Support (CDS) may be provided by proposed concepts. Also, the collection and analysis of high resolution data responsive to detecting an abnormal respiratory event may facilitate tailored diagnostics. Proposed approaches may focus on event-dependent acquistion of respiration data to enable efficient and accurate abnormal respiratory event monitoring. By way of example, this may provide for: reduced subject administration or interrogation; improved respiratory disease management; and iterative improvement of subject/event-specific diagnostics, treatment and management.

In some embodiments, the first detection frequency may be within the range of 0 Hz to 50 Hz, and the second detection frequency may be within the range of 50 Hz to 2000 Hz. Although the top end of such an exemplary range for the second detection frequency may be 2000 Hz, in case of limited battery or processing power, the second detection frequency may be set to a lower value such as 200 Hz. Ignoring the battery and processing requirement, the sampling frequency in a post abnormal respiratory event time window (e.g. post cough window) could be set as high as 2 kHz, or even 5 kHz, so as to provide more detailed and clinically-valuable respiratory event characterization.

By way of example, detecting an abnormal respiratory event based on the first respiration data may comprise: processing the first respiration data with an algorithm configured to detect at least one of: a cough; a wheeze; shortened breath; dyspnea; and orthopnea, trepopnea, platypnea, Cheyne-Stokes respiration, extended period of hyperventilation, tachypnea, and symptoms of exacerbation of chronic obstructive pulmonary disease.

Some embodiments may also include controlling a function of the sensor system based on the second respiration data. Proposed embodiments may thus include a concept of modifying an operation or behavior of the sensor system, based on the respiration data acquired at the second, higher frequency. For instance, the display of information by a wearable sensor device may be controlled so as to display a determined parameter value or characteristic of the subject's respiration. By way of further example, an application or notification may be automatically provided to the subject or their caregivers in response to the second respiration data exhibiting a characteristic or value that meets a predetermined requirement. Also, in some embodiments, a function or algorithm performed by a sensor or device of the sensor system may be adapted based on the respiration data acquired at the second, higher frequency. For instance, a parameter of a detection or monitoring function provided by the wearable device may be adapted to account for a determined parameter value or characteristic of the subject's respiration.

Embodiments may further comprise analyzing the second respiration data to determine one or more parameters of the detected abnormal respiratory event. For example, analysing the second respiration data may comprise: processing the second respiration data with an algorithm configured to detect at least one of: a cough; a wheeze; shortened breath; dyspnea; and orthopnea, trepopnea, platypnea, Cheyne-Stokes respiration, extended period of hyperventilation, tachypnea, and a symptom of exacerbation of chronic obstructive pulmonary disease. Such ‘second stage’ analysis may use the same class of algorithms (statistical and structured machine learning models, sequence-based stochastics models, etc.) as the ‘first stage’ of analysis with the difference that the inputs to the algorithm are data streams sampled at the higher frequency. These inputs may be meta-features representing the high-frequency data or the algorithm may use the high-frequency data streams directly. Further, the algorithm may use output of the first-stage algorithm (i.e. results from processing the first respiration data) as an input.

Embodiments may use both data streams (i.e. first and second respiration data) from the sensor system along with contextual information (time of the day, seasonality information) and information from subject's profile (e.g. current location, chronic respiratory conditions, history of respiratory events, user's medication) to automatically detect respiratory complications. One of more notifications may be provided (e.g. to a medical professional and/or a care-giver) in the event of a detected respiratory complication.

Some embodiments may further comprise determining a position of the sensor system relative to the subject's body. Analyzing the second respiration data to determine one or more parameters of the detected abnormal respiratory event may then be based on the determined position of the sensor system. In this way, embodiments may be configured to account for a specific position of a sensor, thus making data analysis more accurate (e.g. by adjusting the analysis according to the specific context of the sensor).

Proposed embodiments may also further comprise generating a control signal for controlling a function of a device based on the determined one or more parameters of the detected abnormal respiratory event. Embodiments may thus include a concept of controlling an operation or behavior of a supplementary/additional device based on the respiration data acquired at the second, higher frequency. For instance, the display of information by a portable computing device (such as a smartphone or tablet computer) may be controlled so as to display a determined parameter value or characteristic of the subject's respiration. By way of further example, an application or notification may be automatically provided to the subject or their caregivers via a portable computing device and/or portable notification device in response to the second respiration data exhibiting a characteristic or value that meets a predetermined requirement.

Purely by way of example, the subject may comprise a patient. Embodiments may therefore be used to monitor a patient within a hospital room that already comprises conventional patient monitoring system for example. Illustrative embodiments may be utilized in many different types of clinical, medical or patient-related environments, such as a hospital, doctor's office, ward, care home, person's home, etc.

According to another aspect, there is provided a computer program product for monitoring abnormal respiratory events of a subject using a sensor system configured to detect respiration of the subject and generate respiration data representative of the detected respiration, wherein the computer program product comprises a computer-readable storage medium having computer-readable program code embodied therewith, the computer-readable program code configured to perform all of the steps of a proposed embodiment.

Thus, there may also be provided a computer system comprising: a computer program product according to proposed embodiment; and one or more processors adapted to perform a method according to a proposed concept by execution of the computer-readable program code of said computer program product.

According to still another aspect of the invention, there is provided a system for monitoring abnormal respiratory events of a subject using a sensor system configured to detect respiration of the subject and generate respiration data representative of the detected respiration. The system comprises: a controller configured to control the sensor system to detect respiration of the subject at a first detection frequency; an interface component configured to obtain first respiration data from the sensor system, the first respiration data being representative of respiration of the subject detected at the first detection frequency; and a data analysis component configured to detect an abnormal respiratory event of the subject based of the first respiration data, wherein the controller is configured to, responsive to the data analysis component detecting an abnormal respiratory event of the subject, control the sensor system to detect respiration of the subject at a second detection frequency, the second detection frequency being higher than the first detection frequency, and wherein the interface component is configured to obtain second respiration data from the sensor system, the second respiration data being representative of respiration of the subject detected at the second detection frequency.

Embodiments may thus provide a system that can automatically monitor a subject's coughing, wheezing or other abnormal respiratory events. Such an embodiment may not require a dedicated/specialized sensor for respiratory tracking. Rather an embodiment may be used in conjucntion with a pre-existing chest-worn sensors, the type of which is used in pendant-based activity/fall trackers, to also monitor a subject's abnormal respiratory events.

The system may be remotely located from a user device. In this way, a user (such as a medical professional) may have an appropriately arranged system that can receive information at a location remotely located from the system for automatic and dynamic monitoring of abnormal respiratory events of a subject. Embodiments may therefore enable a user to monitor a subject using a local system (which may, for example, comprise a portable display device, such as a laptop, tablet computer, mobile phone, PDA, etc.). By way of example, embodiments may provide an application for a mobile computing device, and the application may be executed and/or controlled by a user of the mobile computing device.

The system may further include: a server device comprising for monitoring abnormal respiratory events of a subject; and a client device comprising a user-interface. Dedicated data processing means may therefore be employed for the purpose of for monitoring abnormal respiratory events of a subject, thus reducing processing requirements or capabilities of other components or devices of the system.

The system may further include a client device, wherein the client device comprises the controller, interface component and a display unit. In other words, a user (such as a doctor, caregiver or medical professional) may have an appropriately arranged client device (such as a laptop, tablet computer, mobile phone, PDA, etc.) which controls a sensor system and processes received respiration data in order to monitor abnormal respiratory events of a subject and generate a display control signal. Purely by way of example, embodiments may therefore provide a monitoring system that enables monitoring of one or more environments (each including subjects or patients for example) from a single location, wherein real-time communication between a monitored environment and monitoring user (e.g. nurse or doctor) is provided and can have its functionality extended or modified according to proposed concepts, for example.

It will be understood that processing capabilities may therefore be distributed throughout the system in different ways according to predetermined constraints and/or availability of processing resources.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 is a simplified flow diagram of a method for monitoring abnormal respiratory events of a subject according to an embodiment;

FIG. 2 depicts a simplified block diagram of system for monitoring abnormal respiratory events of a subject according to an embodiment; and

FIGS. 3 depicts an exemplary embodiment for monitoring abnormal respiratory events of a subject.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. The mere fact that certain measures are recited in mutually different dependent claims does not indicate that a combination of these measures cannot be used to advantage.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. It should be understood that the Figures are merely schematic and are not drawn to scale. It should also be understood that the same reference numerals are used throughout the Figures to indicate the same or similar parts.

Proposed is an approach for monitoring abnormal respiratory events of a subject which controls a sensor system to acquire data at different rates (e.g. different sampling frequencies), wherein the data acquisition rate is controlled in response to detecting an abnormal respiratory event. Embodiments may control the sensor system to acquire data at a first, low frequency from which an abnormal respiratory event (such as a cough or wheeze) of the subject may be detected. Then, in response to detecting an abnormal respiratory event, the sensor system may be controlled to acquire data at a second, higher frequency so as to facilitate more detailed analysis and/or monitoring of the subject's respiration.

Such proposals may thus facilitate reduced power consumption by the sensor system whilst still ensuring that accurate, high-resolution data is acquired when necessary or appropriate.

Embodiments of the present invention are therefore directed toward monitoring abnormal respiratory events of a subject, and aim to make use of existing or conventional sensor systems.

As a result, embodiments may facilitate routine, unobtrusive, and in-home tracking of respiratory complications using conventional monitoring devices (such as a chest-worn pendant, the type of which used for mobility tracking and fall detection in PERS systems). By way of example, embodiments may use respiration data provided from accelerometer signals (and/or barometer and gyroscope signals) collected by a chest-worn pendant.

Proposed embodiments may therefore be particularly relevant for use with subjects suffering from with respiratory-limiting conditions such as COPD, because they may enable an additional level of monitoring without the need for new and/or additional wearable sensors for example.

By way of example only, illustrative embodiments may be utilized in many different types of clinical, medical or subject-related environments, such as a hospital, doctor's office, ward, care home, person's home, etc. For instance, embodiments may be employed to monitor a patient in a hospital room. Embodiments may facilitates the prompt notification of caregivers and/or prompt delivery of urgent care when needed, leading to improved quality of life.

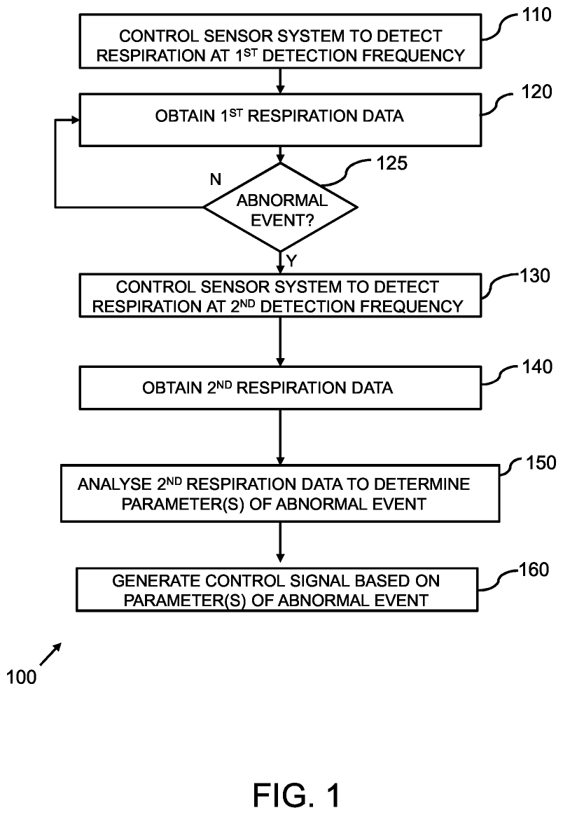

FIG. 1 is a simplified flow diagram of a computer-implemented method for monitoring abnormal respiratory events of a subject. The method is configured for use with a sensor system that is adapted to detect respiration of the subject and to generate respiration data representative of the detected respiration.

The method begins with step 110 of controlling the sensor system to detect respiration of the subject at a first detection frequency. This results in the sensor system generating (first) respiration data representative of the respiration detected at the first detection frequency. In this example, the first detection frequency is within the range of 0 Hz to 50 Hz. Put another way, the sensor system is controlled to generate first respiration data representative of respiration of the subject that is detected at a low frequency (i.e. sampled at a low sampling rate), e.g. once every few seconds, once a second, a few times per second, or tens of times per second.

The method then comprises step 120 of obtaining generated first respiration data from the sensor system, the first respiration data being representative of respiration of the subject detected at the first detection frequency. This may, for example, comprise receiving the first respiration data via a wireless communication link and/or via the Internet.

Based on the first respiration data, it is determined whether or not an abnormal respiratory event of the subject has occurred in step 125. In other words, step 125 comprises detect an abnormal respiratory event based on the first respiration data.

By way of example, step 125 of detecting an abnormal respiratory event comprises processing the first respiration data with an algorithm configured to detect at least one of: a cough; a wheeze; shortened breath; dyspnea; and orthopnea, trepopnea, platypnea, Cheyne-Stokes respiration, extended period of hyperventilation, tachypnea, and a symptom of exacerbation of chronic obstructive pulmonary disease. Such a detection algorithm may use a moving-window approach evaluating the signal energy or power in a window and detecting an abnormal respiratory event if the signal energy or power is above or below an expert-authored threshold. Alternatively, the detection algorithm may comprise a mapping between signal characteristics and a set of adverse respiratory events (e.g., heightened cough). The mapping may, for example, be learnt using a logistic regression or similar statistical models, structured machine learning models (e.g. gradient boosted regression trees), or sequence-based machine learning models that incorporate temporal information (e.g. hidden Markov models or recurrent neural network models).

If no abnormal respiratory event is detected in step 125, the methods returns to step 120 of obtaining first respiration data in order to continue monitoring for the occurrence of an abnormal respiratory event.

If an abnormal respiratory event is detected in step 125, the method proceeds to step 130. In step 130, the sensor system is controlled to detect respiration of the subject at a second detection frequency, the second detection frequency being higher than the first detection frequency. In this example, the second detection frequency is within the range of 50 Hz to 2000 Hz. In this way, the sensor system is controlled to generate second respiration data representative of respiration of the subject that is detected at a high frequency (i.e. sampled at a high sampling rate, relative to the low sampling rate), e.g. a hundred times per second, many hundreds of times per second, or thousands of times per second. This results in the sensor system generating (second) respiration data representative of the respiration detected at the second detection frequency.

The method then comprises step 140 of obtaining second respiration data from the sensor system, the second respiration data being representative of respiration of the subject detected at the second detection frequency. Again, this may, for example, comprise receiving the second respiration data via a wireless communication link and/or via the Internet.

Subsequently, the method proceeds to step 150. Step 150 comprises analyzing the second respiration data to determine one or more parameters of the detected abnormal respiratory event. Here, analyzing the second respiration data comprises processing the second respiration data with an algorithm configured to detect at least one of: a cough; a wheeze; shortened breath; dyspnea; and orthopnea, trepopnea, platypnea, Cheyne-Stokes respiration, extended period of hyperventilation, tachypnea, and a symptom of exacerbation of chronic obstructive pulmonary disease. Thus, such analysis may use the same class of algorithms (statistical and structured machine learning models, sequence-based stochastics models) as the analysis of the first respiration data with the difference that the inputs to the algorithm are data streams sampled at the higher frequency.

Further, statistical and structured machine learning models may learn a mapping between meta-features characteristic of the observed signals and likelihoods of respiratory events (feature-based models). Sequence-based models, such as a gated recurrent neural network or continuous hidden Markov models, directly capture temporal progression of the data streams from sensors and detect incidences of abnormal respiratory events through examining deviations from healthy respiratory patterns for the user (temporal models). Alternatively, a hybrid model combining a temporal model and a feature-based model may also be used that receives sequences of data streams from sensors along with information on subject profile (e.g., current location, chronic respiratory conditions, history of respiratory events, time of the day, seasonality information, user's medication) and estimates corresponding risk probabilities of adverse respiratory event and the type of most likely event.

Finally, based on the determined one or more parameters of the detected abnormal respiratory event, a control signal for controlling a function of a device is generated in step 160. In this example, the control signal is adapted to control the display of information by a portable computing device. In this way, the portable computing device can be controlled to display the determined parameter value or characteristic of the subject's respiration. The control signal may also control the portable computing device to provide a notification if the determined parameter value or characteristic of the subject's respiration meets a predetermined requirement (e.g. exceeds an acceptable threshold).

From the above description, it will be appreciated that the embodiment of FIG. 1 provides an approach for monitoring abnormal respiratory events of a subject in which a sensor system is controlled to acquire respiration data at different rates (e.g. different sampling frequencies). Switching from a first, low frequency data acquisition rate to a second, higher frequency data acquisition is undertaken in response to detecting an abnormal respiratory event. In this way, respiration data of higher resolution may be obtained for a time window immediately following the occurrence of an abnormal respiratory event, thus enabling detailed and accurate analysis of the subject's respiration following the abnormal respiratory event. Processing power and resource of the sensor system may thus be preserved only for when an abnormal respiratory event is detected and accompanying data of increased resolution may be valuable.

In the embodiment of FIG. 1, it is detailed that step 150 comprises analyzing the second respiration data to determine one or more parameters of the detected abnormal respiratory event. However, it is noted that, in some embodiments, the algorithm in step 150 may also use, as an extra input, results from processing the first respiration data. Embodiments may thus use both data streams (i.e. first and second respiration data) from the sensor system along with contextual information (time of the day, seasonality information) and information from subject's profile (e.g. current location, chronic respiratory conditions, history of respiratory events, user's medication) to automatically detect respiratory complications.

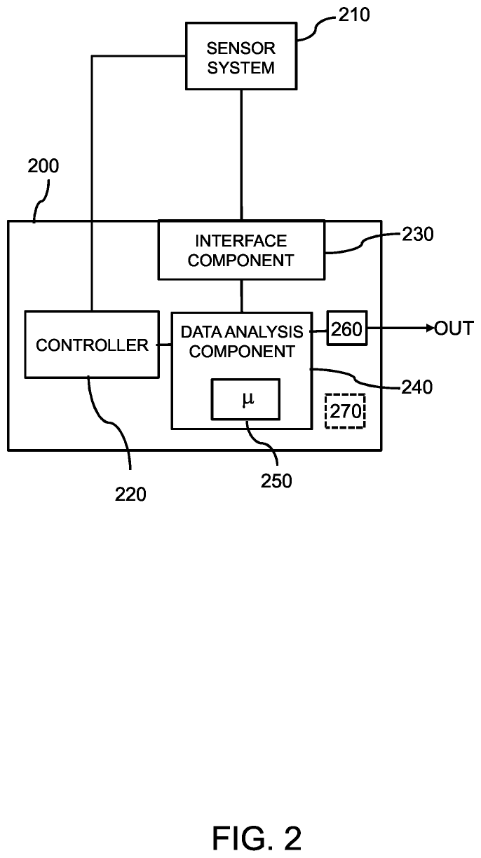

By way of further illustration of the proposed concept(s), a system for monitoring abnormal respiratory events of a subject according to an embodiment will be now be described with reference to FIG. 2.

FIG. 2 depicts a simplified block diagram of system 200 monitoring abnormal respiratory events of a subject according to an embodiment. FIG. 2 also depicts a sensor system 210 that is configured to detect respiration of the subject and generate respiration data representative of the detected respiration.

In this example, the sensor system 210 comprises a conventional activity tracking device (comprising an accelerometer) that is worn by the subject around the sternum (like a belt).

The system 200 comprises a controller 220 that is configured to control the sensor system 210 to detect respiration of the subject at a first detection frequency in the range of 10 Hz to 25 Hz.

An interface component 230 of the system 200 is configured to obtain first respiration data from the sensor system 210, the first respiration data being representative of respiration of the subject detected at the first detection frequency. In this example, the first respiration data comprises values of detected movement of the sternum of the subject, the values being detected at the first detection frequency.

A data analysis component 240 of the system 200 is then configured to detect an abnormal respiratory event of the subject based of the first respiration data. In this example, the data analysis component 240 comprises a (micro-)processor 250 that is configured to process the first respiration data with an algorithm configured to detect at least one of: a cough; a wheeze; shortened breath; dyspnea; trepopnea, platypnea; Cheyne-Stokes respiration, extended period of hyperventilation, tachypnea, orthopnea, and symptoms of exacerbation of chronic obstructive pulmonary disease. Here, one of many known algorithms for detecting a cough from detected movement of the subject's sternum may be employed, wherein a cough may be identified by a pattern of detected movement of the subject's sternum.

Responsive to the data analysis component 240 detecting an abnormal respiratory event of the subject based of the first respiration, the controller 220 is configured to control the sensor system 210 to detect respiration of the subject at a second detection frequency, the second detection frequency being higher than the first detection frequency. In this example, the second detection frequency is in the range of 100 Hz to 1 kHz.

The interface component 230 is configured to obtain second respiration data from the sensor system, the second respiration data being representative of respiration of the subject detected at the second detection frequency. Thus, in this example, the second respiration data comprises values of detected movement of the sternum of the subject, the values being detected at the second, higher detection frequency.

The data analysis component 240 is then further configured to analyze the second respiration data to determine one or more parameters of the detected abnormal respiratory event.

The system 200 also comprises an output interface 260 adapted to generate a control signal OUT for controlling a function of a device based on the determined one or more parameters of the detected abnormal respiratory event. In this example, the output interface 260 generates a control signal OUT for instructing one or more devices to generate a notification if the determined one or more parameters of the detected abnormal respiratory event meet a predetermined requirement (e.g. exceed a threshold). This may, for example, be used to alert a medical professional and/or caregiver about a respiratory complication experience by the subject.

By way of further example, the system 200 may also include a positioning unit 270 that is configured to determine a position of the sensor system 210 relative to a particular part of the subject's body (e.g. sternum). The data analysis component 240 may then be further configured to determine one or more parameters of the detected abnormal respiratory event taking account of the determined position of the sensor system. In this way, the data analysis component 240 may account for a specific position of the sensor system 210, thus making data analysis more accurate (e.g. by adjusting the analysis according to the specific positioning of the sensor).

To further illustrate the proposed concepts, the main components of an exemplary embodiment may be summarized as follows:
(i) A chest-worn sensor package for continuous cough tracking;
(ii) A cough detection module (i.e. data analysis component);
(iii) A post-cough data acquisition module (e.g. windowed high-frequency tracking);
(iv) An classification module to further evaluate the type and intensity of detected coughs (i.e. cough assessment) based on the acquired post-cough data; and
(v) A communication module adapted to communicate the evaluation results to relevant databases and caregivers.

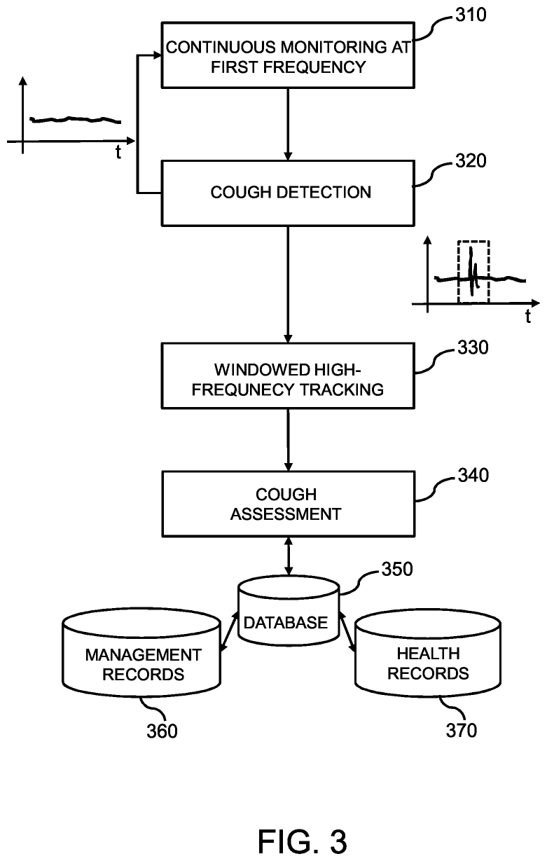

By way of yet further illustration of the proposed concept(s), an exemplary embodiment for monitoring abnormal respiratory events of a subject will be now be described with reference to FIG. 3.

In this exemplary embodiment, a chest-worn sensor package is employed. More specifically, the sensor package comprises a Personal Emergency Response System (PERS) chest-worn pendant equipped with an accelerometer (with at least two axes) that continuously tracks movements of the subject wearing the pendant. It is proposed that coughing and other abnormal respiratory event may be manifested as skin vibrations in the chest and abdomen areas generating motion (and acoustic pressure waves) measurable by an accelerometer (and microphones) positioned on chest (pendant or skin-attached sensor). In a first mode (Step 310), the sensor package detects movement values at a low-frequency rate (e.g. <50 Hz). Alternatively, or additionally, the sensor package may include one or more microphones. The microphone(s) may be activated to scan surroundings for acoustic/prosodic voiced and also unvoiced (silent) portions of the acoustic observations succeeding a cough event.

A cough detection module (e.g. combined interface component and data analysis component) receives low-frequency signals collected by the sensor package in step 310 and detects coughing episodes (based on signals characteristic of coughs that are distinct in time and frequency features from those associated with ambulatory and gross body movements) (Step 320). The cough detection module uses a threshold-based approach that tracks windowed signal energy to detect coughing events. Furthermore, cough detection module distinguishes between a cough and other types of vocalizations (speech) and heart sounds based on temporal and frequency spectral features most salient to coughs.

By default, the wearable senor package acquires data in the first mode (Step 310) at a low-frequency (<50 Hz). A post-cough acquisition module is initiated when a cough event is detected. This module activates a higher-frequency accelerometer data acquisition (>200 Hz) for 30 seconds at a time. The sampling frequency in the 30-sec post-cough window could be set to a higher frequency (e.g., 2 kHz) for more detailed and clinically-valuable cough type and intensity characterization. Thus, responsive to detecting a cough, a second mode (Step 330) is entered wherein windowed high-frequency tracking is enabled.

The data acquired in the second mode (in step 330) is processed to assess cough severity and implement a more detailed cough assessment (Step 340).

Both, the cough detection (320) and the post-cough data acquisition (330) may also execute a signal segmentation processes to isolate segments corresponding to coughs from those corresponding to gross body movements and other type of vocalizations. In addition to cough-specific segments, this feature may identify signals associated with abnormal breathing, labored and noisy breathing, wheezing, extended period of hyperventilation, tachypnea, cheyne-Stokes respiration, expiratory grunting, and swallowing aspiration. The segmentation feature could employ a windowed feature-based approach that tracks the changes in time-frequency features and marks a segment once a significant change in these features is detected or features characteristic of the event of interest are observed within a window.

A communication module may be configured to communicate detected respiratory distress along with its characteristics to caregivers. The level and type of information can be tailored to caregiver (family members vs professional clinical caregivers). The information can be used by the caregiver to deliver urgent and the right level of care (e.g., medication administration). Furthermore, this information (at different level of details from logs of events to a detailed characterization) will be stored for assessment of user's health in relevant databases, such as a main database 350, a population management database 360, and a health records database 370, which can over time be used for diagnostic purposes and early detection of exacerbation in respiratory conditions (for example in step 340).

Proposed embodiment may also employ a classification module to further assess the type and intensity of detected coughs. Such a classification module may receive isolated signal sequences and classify them into different cough types (e.g. wet, dry). For this, the classification module can use a gated recurrent neural network architecture and/or a feature-based classification approach that receives sequences of isolated signal, identifies and attends to features characteristic of the class of a respiratory complication episode.

It will be understood that the disclosed methods are computer-implemented methods. As such, there is also proposed a concept of a computer program comprising code means for implementing any described method when said program is run on a processing system.

The skilled person would be readily capable of developing a processor for carrying out any herein described method. Thus, each step of a flow chart may represent a different action performed by a processor, and may be performed by a respective module of the processing processor.

From the above-described embodiments, it will be understood that there is proposed a concept for monitoring abnormal respiratory events of a subject in which a sensor system is controlled to acquire data at different rates (e.g. different sampling frequencies). Switching from a first, low frequency data acquisition rate to a second, higher frequency data acquisition is executed responsive to detecting an abnormal respiratory event. Processing power and resource of the sensor system may thus be preserved only for when an abnormal respiratory event is detected. Accordingly, proposed embodiments may provide concepts for in-home, routine, objective, and low-burden detection of respiratory complications and evaluation of the type and intensity of detected complications. In one example, it is proposed to leverage a sensor package worn on the chest area (without necessarily being mechanically attached to the body for example using adhesives, rather the sensor package may be in the form of pendant sitting on the chest or abdomen area of subject's body) that includes a dual axial accelerometer, but could also include a gyroscope, a magnetometer, or a barometer. The system could also include one or more microphones.

Thus, not only may embodiments faciliate the detection of episodes of respiratory complications, but embodiments may also facilitate detailed assessment of detected complication episodes to identify the type and intensity of the complications. Therefore, the proposed system provides an additional level of monitoring without the need for adding new or additional monitoring sensors/devices or changing subject's behaviour.

As discussed above, the system makes use of a processor to perform the data processing. The processor can be implemented in numerous ways, with software and/or hardware, to perform the various functions required. The processor typically employs one or more microprocessors that may be programmed using software (e.g. microcode) to perform the required functions. The processor may be implemented as a combination of dedicated hardware to perform some functions and one or more programmed microprocessors and associated circuitry to perform other functions.

Examples of circuitry that may be employed in various embodiments of the present disclosure include, but are not limited to, conventional microprocessors, application specific integrated circuits (ASICs), and field-programmable gate arrays (FPGAs).

In various implementations, the processor may be associated with one or more storage media such as volatile and non-volatile computer memory such as RAM, PROM, EPROM, and EEPROM. The storage media may be encoded with one or more programs that, when executed on one or more processors and/or controllers, perform the required functions. Various storage media may be fixed within a processor or controller or may be transportable, such that the one or more programs stored thereon can be loaded into a processor.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. If the term “adapted to” is used in the claims or description, it is noted that the term “adapted to” is intended to be equivalent to the term “configured to”. Any reference signs in the claims should not be construed as limiting the scope.

1. A computer-implemented method for monitoring abnormal respiratory events of a subject using a sensor system configured to detect respiration of the subject and generate respiration data representative of the detected respiration, the method comprising:
controlling the sensor system to detect respiration of the subject at a first detection frequency;
obtaining first respiration data from the sensor system, the first respiration data being representative of respiration of the subject detected at the first detection frequency;
detecting an abnormal respiratory event of the subject based of the first respiration data;
responsive to detecting an abnormal respiratory event of the subject, controlling the sensor system to detect respiration of the subject at a second detection frequency, the second detection frequency being higher than the first detection frequency; and
obtaining second respiration data from the sensor system, the second respiration data being representative of respiration of the subject detected at the second detection frequency.

2. The method of claim 1, wherein the first detection frequency is within the range of 0 Hz to 50 Hz, and wherein the second detection frequency is within the range of 50 Hz to 2000 Hz.

3. The method of claim 1, wherein detecting an abnormal respiratory event based on the first respiration data comprises:
processing the first respiration data with an algorithm configured to detect at least one of: a cough; a wheeze; shortened breath; dyspnea; and orthopnea, trepopnea, platypnea, Cheyne-Stokes respiration, extended period of hyperventilation, tachypnea, and a symptom of exacerbation of chronic obstructive pulmonary disease.

4. The method of claim 1, further comprising:
controlling a function of the sensor system based on the second respiration data.

5. The method of claim 1, further comprising:
analysing the second respiration data to determine one or more parameters of the detected abnormal respiratory event.

6. The method of claim 5, wherein analysing the second respiration data comprises:
processing the second respiration data with an algorithm configured to detect at least one of: a cough; a wheeze; shortened breath; dyspnea; and orthopnea, trepopnea, platypnea, Cheyne-Stokes respiration, extended period of hyperventilation, tachypnea, and a symptom of exacerbation of chronic obstructive pulmonary disease.

7. The method of claim 5, further comprising determining a position of the sensor system relative to the subject's body, and wherein analysing the second respiration data to determine one or more parameters of the detected abnormal respiratory event is based on the determined position of the sensor system.

8. The method of claim 5, further comprising:
generating a control signal for controlling a function of a device based on the determined one or more parameters of the detected abnormal respiratory event.

9. A computer program comprising computer program code means which is adapted, when said computer program is run on a computer, to implement the method of any of claim 1.

10. A system for monitoring abnormal respiratory events of a subject using a sensor system configured to detect respiration of the subject and generate respiration data representative of the detected respiration, the system comprising:
a controller configured to control the sensor system to detect respiration of the subject at a first detection frequency;
an interface component configured to obtain first respiration data from the sensor system, the first respiration data being representative of respiration of the subject detected at the first detection frequency; and
a data analysis component configured to detect an abnormal respiratory event of the subject based of the first respiration data,
and wherein the controller is configured to, responsive to the data analysis component detecting an abnormal respiratory event of the subject, control the sensor system to detect respiration of the subject at a second detection frequency, the second detection frequency being higher than the first detection frequency,
and wherein the interface component is configured to obtain second respiration data from the sensor system, the second respiration data being representative of respiration of the subject detected at the second detection frequency.

11. The system of claim 10, wherein the data analysis component comprises:
a processor configured to process the first respiration data with an algorithm configured to detect at least one of: a cough; a wheeze; shortened breath; dyspnea; trepopnea, platypnea; Cheyne-Stokes respiration, extended period of hyperventilation, tachypnea, orthopnea, and symptoms of exacerbation of chronic obstructive pulmonary disease

12. The system of claim 10, wherein the controller is further configured to control a function of the sensor system based on the second respiration data.

13. The system of claim 10, wherein the data analysis component is further configured to analyse the second respiration data to determine one or more parameters of the detected abnormal respiratory event.

14. The system of claim 13, further comprising a positioning unit configured to determine a position of the sensor system relative to the subject's body, and wherein the data analysis component is configured to determine one or more parameters of the detected abnormal respiratory event based on the determined position of the sensor system.

15. The system claim 13, further comprising:
an output interface adapted to generate a control signal for controlling a function of a device based on the determined one or more parameters of the detected abnormal respiratory event.
